(19) United States
(12) Patent Application Publication
(10) Pub. No.: US 2025/0228741 A1
(43) Pub. Date: Jul. 17, 2025

(54) OPENING MANAGEMENT SYSTEM AND OPENING DETECTION DEVICE

(71) Applicants: UACJ CORPORATION, Tokyo (JP); UACJ FOIL CORPORATION, Tokyo (JP)

(72) Inventors: Tomohiro MAEDA, Tokyo (JP); Atsushi KOISHIKAWA, Tokyo (JP); Kenji NOSE, Tokyo (JP)

(73) Assignee: UACJ CORPORATION, Tokyo (JP)

(21) Appl. No.: 18/848,435

(22) PCT Filed: Mar. 30, 2023
(86) PCT No.: PCT/JP2023/013159

§ 371 Date: Apr. 9, 2025

(30) Foreign Application Priority Data
Mar. 31, 2022 (JP) 2022-059906

(51) Int. Cl. A61J 1/14; G16H 20/10

(52) U.S. Cl. CPC A61J 1/14; G16H 20/10

(57) ABSTRACT

An opening management system 10 includes a communication section 61 configured to communicate with an opening detection device 40 that is attached to a packaging material 20 and detects an opening status of the packaging material 20, and an information generation section 62 configured to generate setting information with which a predefined process regarding opening detection is performed by the opening detection device 40.

### TECHNICAL FIELD

The present technology relates to an opening management system and an opening detection device.

### BACKGROUND ART

To avoid a failure to take a medicine, a management system that informs a patient of a medication time has been known and one example of such a system is disclosed in Patent Document 1. In the medication management system described in Patent Document 1, schedule information including specified medication dates is sent from an information transmission device in a pharmacy via an email. The sent schedule information is received by an image device (specifically, a television receiver) and the television receiver performs an informing process to inform the medication. More specifically, the television receiver displays a caption of the schedule information or displays the schedule information with the electronic program guide. With the medication management system described in Patent Document 1, by informing a patient with using a commonly used television receiver, the patient is informed surely and effectively and the failure to take a medicine can be avoided.

### PRIOR ART DOCUMENT

### Patent Document

Patent Document 1: Japanese Unexamined Patent Application Publication No. 2007-74068

### Problem to be Solved by the Invention

However, the medication management system described in Patent Document 1 is configured to inform based on the schedule information regardless whether a medicine is taken or not. It cannot be known which medicine is taken by the patient. Therefore, the caption continues to be displayed on the television receiver for a certain time even after the medicine is taken. As a result, a medicine may be taken again erroneously even after the medication is finished or a medicine that is taken may not be the specified one that is to be taken.

### SUMMARY OF THE INVENTION

The present technology was made in view of the above circumstances. An object is to provide an opening management system that can inform based on an opening status and an opening detection device.

### Means for Solving the Problem

Means for solving the problem is described below.
<1> An opening management system includes a communication section configured to communicate with an opening detection device that is attached to a packaging material and detects an opening status of the packaging material, and an information generation section configured to generate setting information with which a predefined process regarding opening detection is performed by the opening detection device.
<2> In the opening management system according to <1>, the setting information includes detection time at which the opening detection device performs the opening detection, and the predetermined process includes detecting the opening status of the packaging material on or after the detection time and informing if the opening status is an unopened state.
<3> In the opening management system according to <1>or <2>, the setting information includes detection frequency at which the opening detection device performs the opening detection, and the predetermined process includes detecting the opening status of the packaging material at the detection frequency and informing if the opening status is an unopened state.
<4> In the opening management system according to <3>, regular detection in which detection is performed every predetermined time period or continuous detection is set as the detection frequency.
<5> In the opening management system according to any one of <1>to <4>, the setting information includes request frequency at which the opening detection device requests transmission of the setting information.
<6> In the opening management system according to <5>, when the communication section receives a request of the transmission of the setting information from the opening detection device, the communication section transmits the setting information to the opening detection device.
<7> In the opening management system according to any one of <1>to <6>, the setting information includes transmission frequency at which the opening detection device transmits the opening status to the communication section.
<8> In the opening management system according to any one of <1>to <7>, the opening detection device includes a plurality of opening detection devices each of which has ID information, the setting information includes the ID information, and the communication section is configured to communicate with each of the plurality of opening detection devices.
<9> In the opening management system according to any one of <1>to <8>, medicine is stored in the packaging material.
<10> An opening detection device includes a

communication section configured to communicate with the opening management system according to any one of <1>to <9>, and a detection section configured to detect the opening status of the packaging material.
<11> The opening detection device according to <10>further includes an informing section configured to inform when a detection result of the detection section is an unopened state.
<12> In the opening detection device according to <10>or <11>, the detection section is configured to detect the opening status of the packaging material on or after detection time included in the setting information.

### Effects of the Invention

According to the present technology, an open management system and an opening detection device that can inform based on an opening status can be achieved.

### BRIEF DESCRIPTION OF THE DRAWINGS

FIG. 1 a general view of an opening management system according to a first embodiment

FIG. 2 a perspective view of an opening detection device that is attached to a packaging material

FIG. 3 a perspective view of the opening detection device

FIG. 4 a perspective view of a built-in components of the opening detection device

FIG. 5 a perspective view of the opening detection device where a second casing is moved upward

FIG. 6 a magnified perspective view of a right portion of a first casing

FIG. 7 a cross-sectional view taken along I-I line in FIG. 3

FIG. 8 a cross-sectional view taken along II-II line in FIG. 5

FIG. 9 a bottom view of the opening detection device

FIG. 10 a perspective view of the opening detection device

FIG. 11 a perspective view of the opening detection device where the second casing is moved upward

FIG. 12 a magnified perspective view of a portion near first projecting portions and a second projecting portion in FIG. 11

FIG. 13 an exploded perspective view of the packaging material

FIG. 14A a flowchart illustrating a process of the opening management system

FIG. 14B a flowchart illustrating a process of the opening management system

FIG. 15A a flowchart illustrating a process of the opening management system

FIG. 15B a flowchart illustrating a process of the opening management system

FIG. 16 a general view of an opening management system according to a second embodiment

### MODES FOR CARRYING OUT THE INVENTION

### First Embodiment

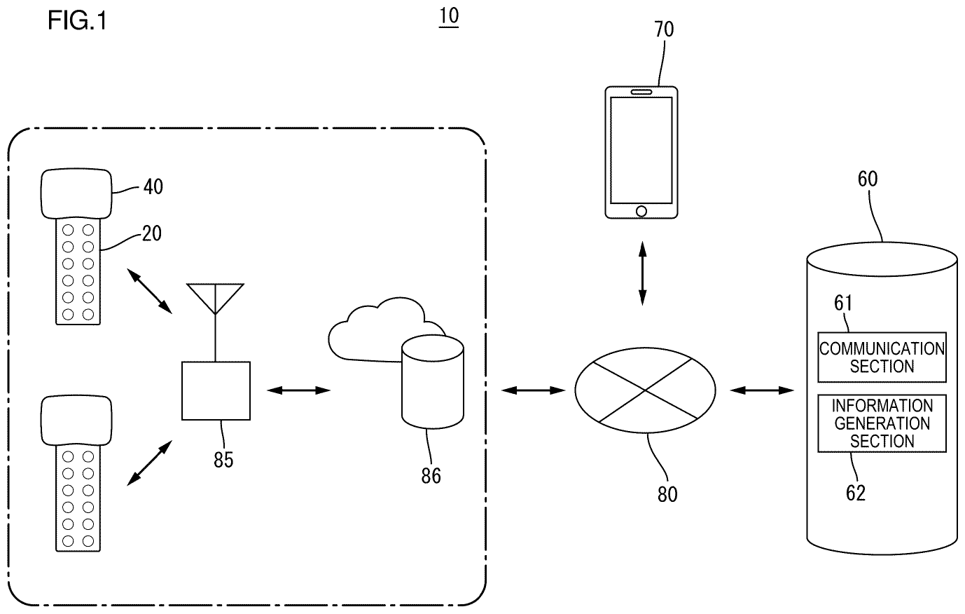

An opening management system 10 according a first embodiment will be described with reference to FIGS. 1 to 15B. An X-axis, a Y-axis and a Z-axis may be present in some of the drawings and each of the axial directions represents a direction represented in each drawing. Direction along the X-axis, the Y-axis, and the Z-axis correspond to the right-left direction, the front-rear direction, and the upper-bottom direction, respectively. However, the directions are defined temporally and may not be limited thereto.

The opening management system 10 is a system for managing medication regarding medicine T based on an opening status of a packaging material 20 that stores the medicine T such as medical tablets (one example of a stored object). According to this embodiment, an opening management system for medicine will be described as an example of the opening management system 10; however, the stored object may not be the medicine T and the opening management system 10 may be widely used for the opening management of the packaging material. As illustrated in FIG. 1, the opening management system 10 includes an opening detection device 40 that is attached to a packaging material 20, a management server (an application server) 60, and an information processing terminal 70. The management server 60 and the information processing terminal 70 are connected to a communication network 80.

As illustrated in FIG. 1, the opening detection device 40 is attached to each packaging material 20. The opening detection device 40 is connected to the communication network 80 via a low power wide area wireless communication (LPWA). The LPWA communication is wireless communication that can achieve long distance communication with small power consumption (for example, transmission distance is 10 km or more with transmission power of 1 W or less). The LPWA communication is known as a basic technology of IoT (internet of things) technology. The LPWA communication service is provided by multiple telecommunication companies and examples of such communication companies include Sigfox (registered trademark), LoRa (registered trademark), and NB-IoT. The opening detection devices 40 are wirelessly connected to a LPWA base station 85 and the base station 85 is connected to a LPWA communication server (LPWA crowd server) 86. The communication server 86 is connected to the communication network 80. Therefore, the opening detection devices 40 can communicate with the management server 60, which is connected to the communication network 80, via the communication server 86. Each of the opening detection devices 40 has its own ID information and the management server 60 can communicate with each of the opening detection devices 40.

The opening detection device 40 needs to be connected to at least one of the management server 60 and the information processing terminal 70. Communication technology other than LPWA may be used as the communication method of the opening detection device 40. For example, the wireless communication technology with low power consumption such as Bluetooth low energy (BLE, registered trademark) is preferable and Wi-Fi or LTE, which are more generally used, may be used as the wireless communication.

As illustrated in FIG. 1, the information processing terminal 70 is a generally-used information processing device such as a smartphone and a computer. It is supposed that the information processing terminal 70 is used by a patient and medical staff of the hospital or the pharmacy who prescribes the medicine T. However, the user of the information processing terminal 70 is not particularly limited (a user of the information processing terminal 70 will be simply referred to as a user). The information processing terminal 70 may include multiple information processing terminals 70 that are used by different users. A user inputs registration information such as a time of taking the medicine T via the information processing terminal 70 and the information processing terminal 70 executes a program for displaying the opening status of the medicine T. In this embodiment, the program is installed in the information processing terminal 70 as an application software (hereinafter, referred to as an application); however, the program may not be installed in the terminal 70 and may be executed via a browser software. This miniaturizes the opening detection device 40 and consumption power of the opening detection device 40 can be reduced.

The management server 60 provides information and processed results in response to the request from the information processing terminal 70 or the opening detection device 40. The management server 60 is configured to perform as a server that executes the program for performing the above functions. In this embodiment, the management server 60 is an example of a server computer illustrated in FIG. 1; however, the management server 60 may be an imaginary one in a computer network in space with distributed computing. The management server 60 may execute processing with artificial intelligence.

As illustrated in FIG. 1, the management server 60 at least includes a communication section 61 that is connected to the communication network 80 and an information generation section 62. The management server 60 can communicate with the information processing terminal 70 via the communication section 61 and communicate with the opening detection devices 40 via the communication server 86. The information generation section 62 generates setting information based on the registration information from the information processing terminal 70. The predefined process regarding the opening detection is performed by the opening detection device 40 according to the setting information. The process performed by the management server 60 will be described later in detail.

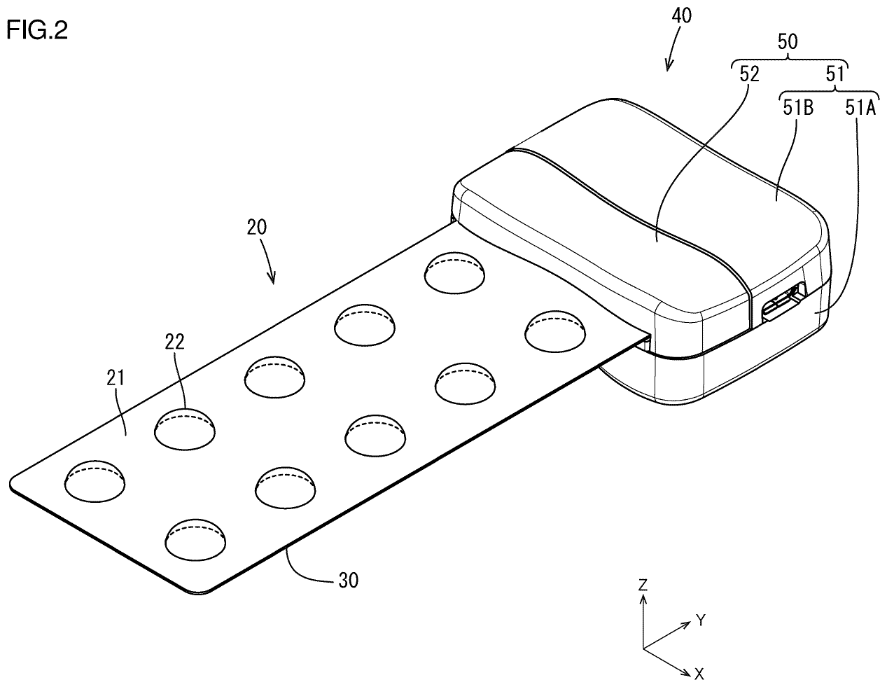

As illustrated in FIGS. 2 to 4, the opening detection device 40 includes connection portions 41 (twelve connection portions 41 in this embodiment), a communication module 42 for the LPWA, a LED 45 (an example of an informing section), a reset button 46 (one example of an operation section), a rechargeable battery 47 (one example of a power supply section), a base board 49, and a casing 50. The connection portions 41, the communication module 42, the LED 45, the reset button 46, and a connector insertion portion 37A via which an external power supply is connected to the rechargeable battery 47 are mounted on the base board 49. The base board 49 on which the components are mounted and the rechargeable battery 47 are arranged in the casing 50. The casing 50 includes a holding portion that sandwiches and releasably holds the packaging material 20.

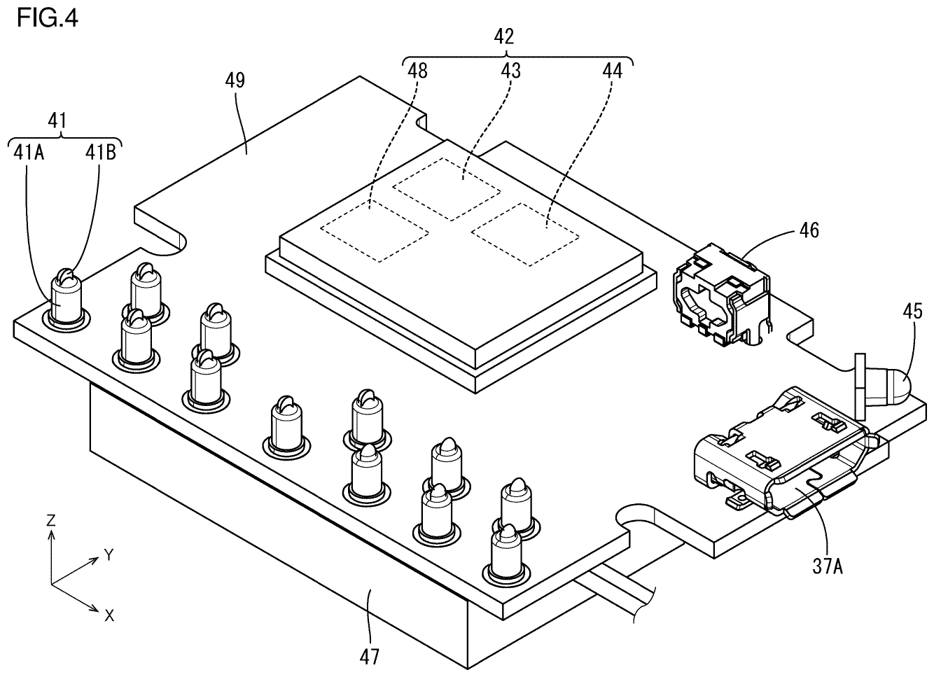

As illustrated in FIG. 4, the connection portion 41 includes a support body 41A and a contact 41B. The support body 41A is mounted on the base board 49 and has a substantially circular columnar shape in this embodiment; however, the shape of the support body 41A is not limited thereto. The contact 41B protrudes upward from an upper surface of the support body 41A (a surface on an opposite side from the base board 49). At least a protruding end of the contact 41B has electrically conductive properties. With the packaging material 20 being set in the opening detection device 40 (FIG. 2), the contacts 41B are contacted with and electrically connected to a circuit pattern 32 of the packaging material 20, which will be described later.

As illustrated in FIG. 4, the communication module 42 includes a communication section 43 (specifically, an antenna), a control section 44 (specifically, a microcomputer), and a storing section 48 (specifically, a ROM and a RAM). The communication section 43 is wirelessly connected to the base station 85 and can communicate with the management server 60 via the communication server 86. The storing section 48 stores control programs and ID information of the opening detection devices 40.

The control section 44 controls each section of the opening detection device 40 based on the control program stored in the storing section 48 and the setting information transmitted from the management server 60. The control section 44 includes a timer having a time counting function. The control section 44 also functions as a detection section detecting opening of the packaging material 20. The control section (the detection section) 44 supplies a current to the connection portions 41 and detects a voltage caused in the circuit pattern 32 of the packaging material 20. The control section 44 compares the detected voltage and a predefined threshold value. If determining that the detected voltage is equal to or smaller than the threshold value, it is determined that the circuit pattern 32 is not broken and the packaging material 20 is in an unopened state. If determining that the detected voltage is greater than the threshold value, it is determined the packaging material 20 is in an opened state where the circuit pattern 32 is broken. The control section 44 can determine the opening status based on the resistance value of the circuit pattern 32 that changes according to the breakage of the circuit pattern 32. The control section 44 may determine the opening status based on a current value with a predefined voltage being applied.

If the detection result (determination result) by the control section 44 is the unopened state, the LED 45 is turned on with steady light (blinking light) with a predefined pattern and it is informed that medicine is not taken yet. The LED 45 is turned on when the opening detection device 40 is connected to an external power supply. The LED 45 is arranged in the casing 50 and the light emitted by the LED 45 passes through the casing 50. More in detail, the casing 50 is formed with a material and a thickness such that the casing 50 can transmit the light emitted by the LED 45. The LED 45 is one example of the informing section. The informing section may be light emitting means other than the LED, sound generating means such as a buzzer, display means displaying a message, or any combination thereof.

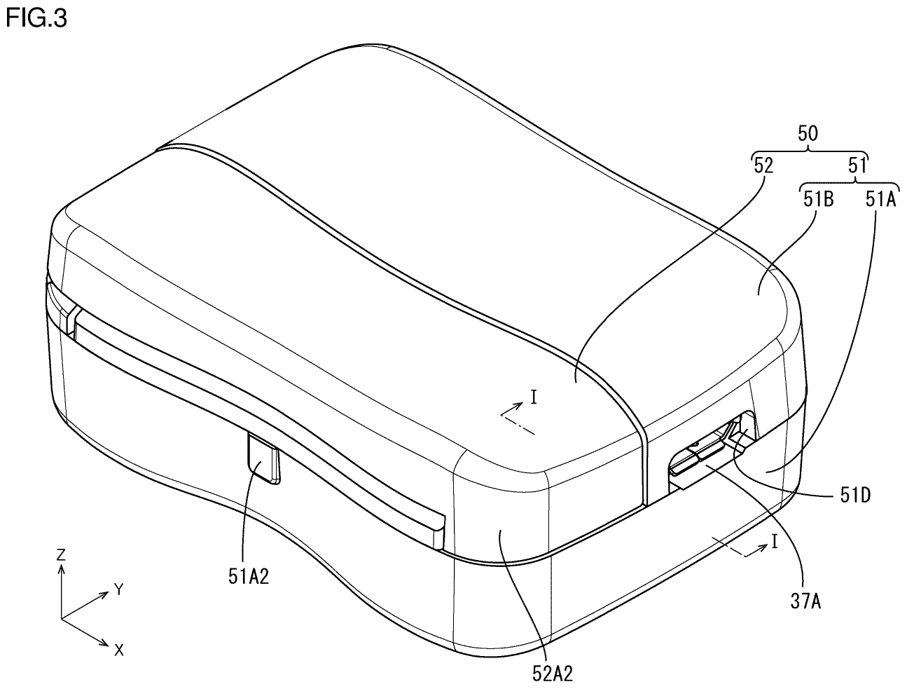

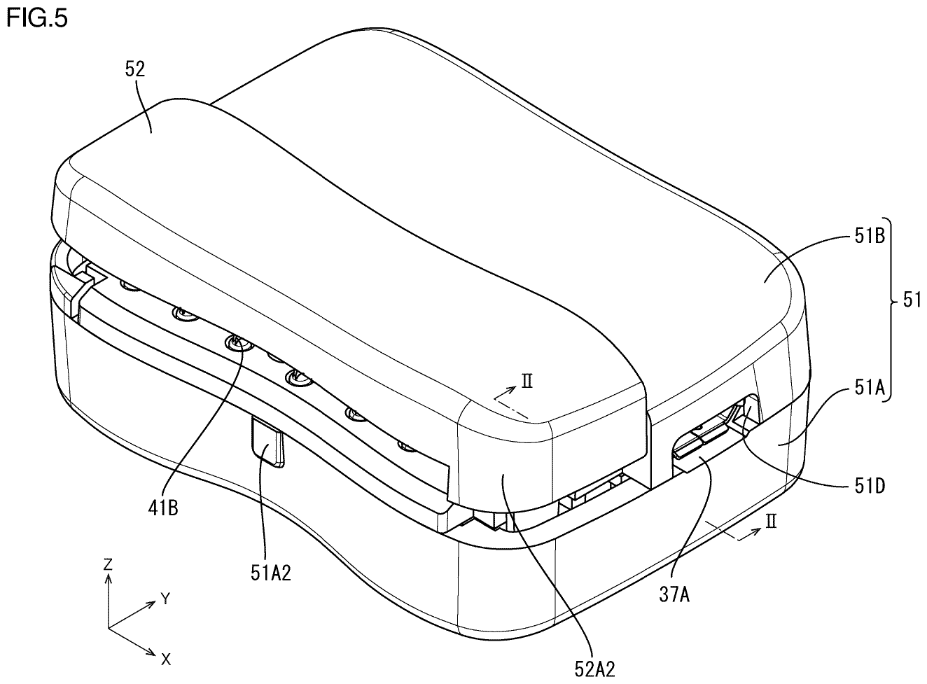

The casing 50 is made of resin and has a laterally elongated cubic shape having round edges as illustrated in FIGS. 3 and 5. The casing 50 includes a first casing 51 and a second casing 52. The first casing 51 corresponds to a most section of the casing 50 and is recessed in a front upper section. The second casing 52 is a cover that covers the recessed front upper section of the first casing 51. The second casing 52 is attached to the first casing 51 so as to be movable in the upper-bottom direction (the Z-axis direction). By moving the second casing 52, as illustrated in FIG. 2, the packaging material 20 is disposed between and held by the first casing 51 and the second casing 52. Namely, the casing 50 includes a holding structure including the first casing 51 and the second casing 52 as the holding portion that holds the packaging material 20. In the following, each component of the casing 50 will be described in detail. The packaging material 20 is not illustrated in the drawings other than FIG. 2 to clearly illustrate the configuration of the casing 50.

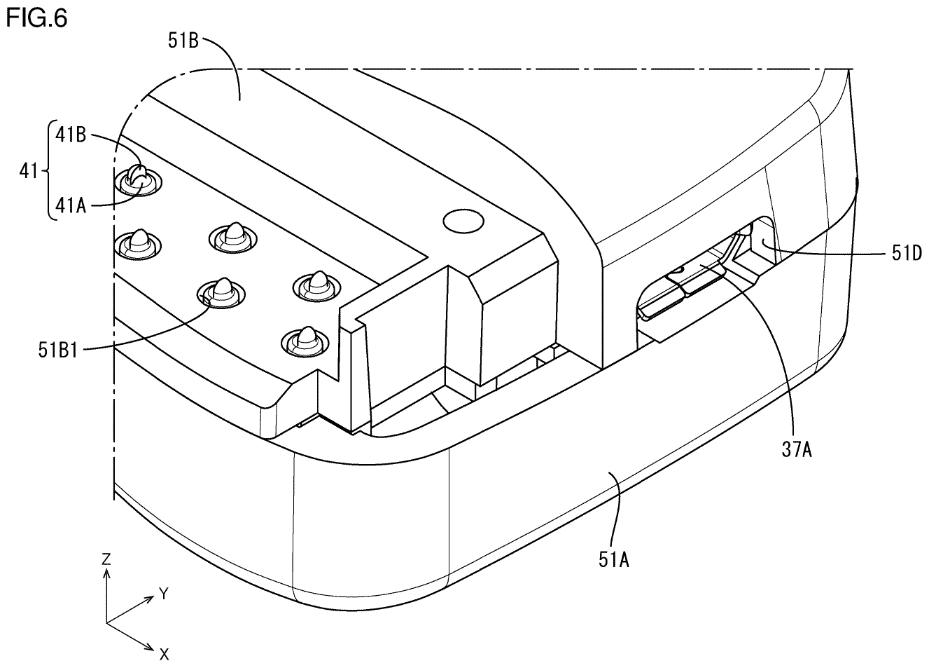

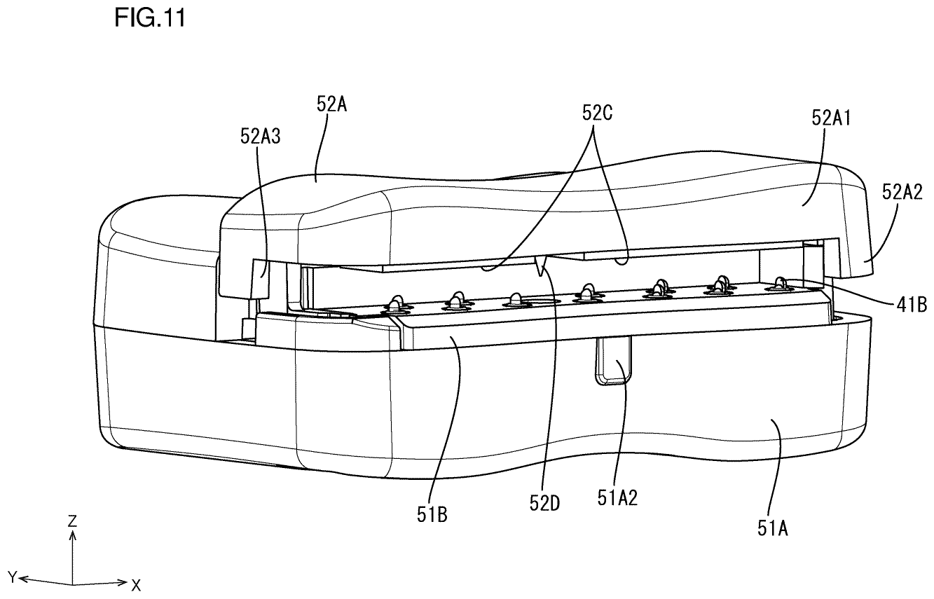

As illustrated in FIGS. 5 to 8, the first casing 51 includes a bottom portion 51A and an upper portion 51B. The bottom portion 51A has a tray shape that opens upward. The upper portion 51B covers the opening of the bottom portion 51A from an upper side. The rechargeable battery 47 is arranged in the bottom portion 51A and the base board 49 is disposed above the rechargeable battery 47. The base board 49 is covered by the upper portion 51B from above. The upper portion 51B includes through holes 51B1 through which the connection portions 41 on the base board 49 pass, respectively. The contacts 41B of the connection portions 41 protrude from the respective through holes 51B1 and are exposed outside (FIG. 6 and FIG. 11).

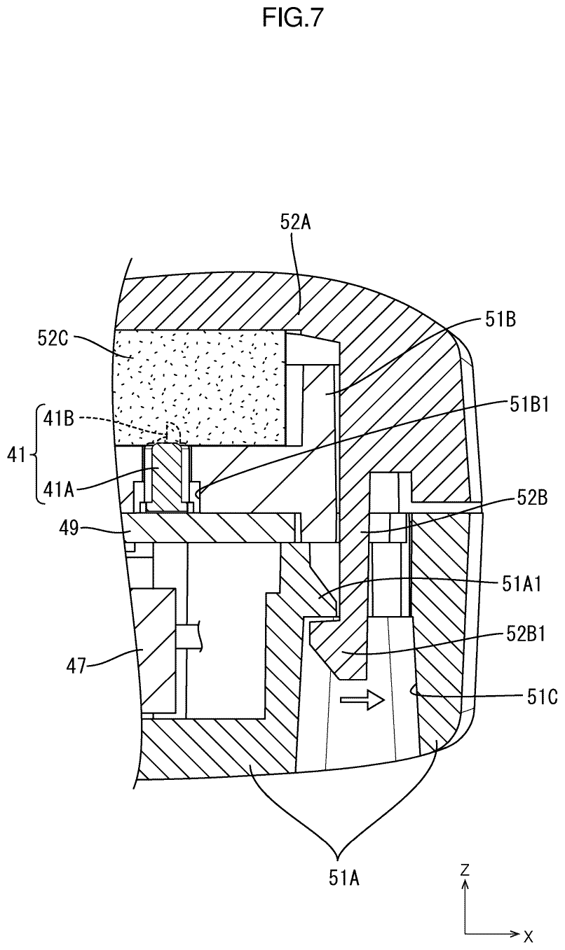

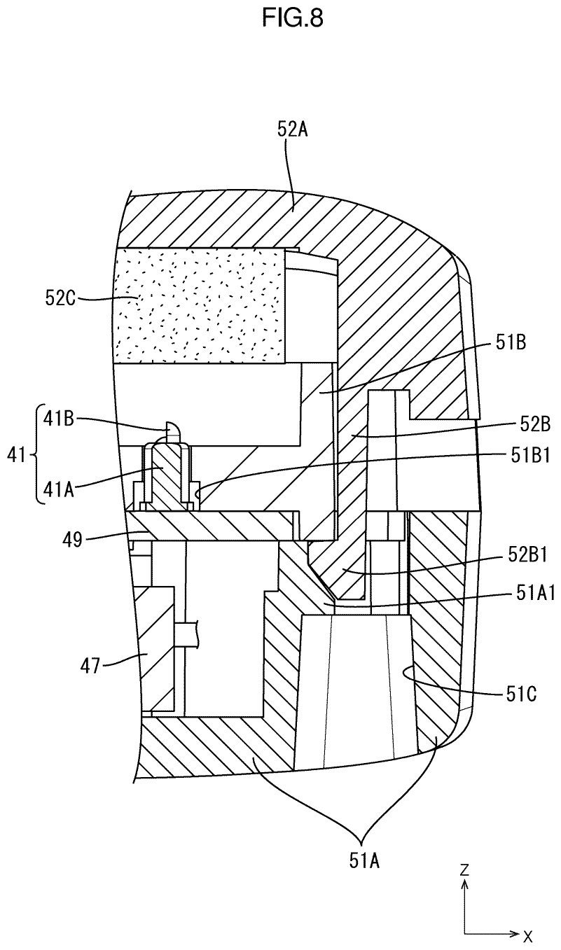

As illustrated in FIGS. 7 to 9, the bottom portion 51A of the first casing 51 includes two first communication holes 51C that receive two fitting portions 52B of the second casing 52, respectively. The first communication holes 51C are formed to overlap the respective fitting portions 52B in a plan view. In the bottom portion 51A of the first casing 51, a portion of an opening edge of the first communication hole 51C protrudes upward and the bottom portion 51A includes a protrusion 51A1 at the protruding end. The protrusion 51A1 is fitted to a protrusion 52B1 of the fitting portion 52B of the second casing 52 (FIG. 7, FIG. 8).

As illustrated in FIG. 5, a positioning mark 51A2 (one example of a positioning portion) is on a front surface of the bottom portion 51A. In this embodiment, the positioning mark 51A2 is a protrusion and formed at a middle of the front surface of the bottom portion 51A with respect to the right-left direction (the X-axis direction). The positioning mark 51A2 is a reference indicator for positioning the packaging material 20 at a predefined position (for example, an end 32A of the circuit pattern 32 of the packaging material 20). With such a configuration, the positioning of the packaging material 20 on the opening detection device 40 is easy and the circuit pattern 32 of the packaging material 20 is surely contacted with the contacts 41B of the opening detection device 40 and the circuit pattern 32 and the opening detection device 40 can be electrically conductive.

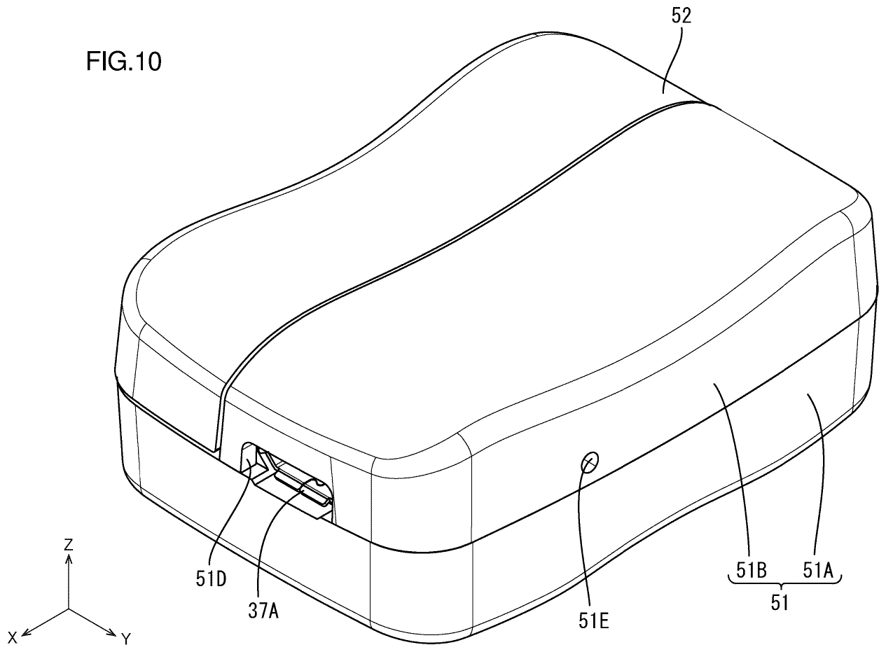

As illustrated in FIG. 5, the first casing 51 includes an insertion hole 51D on a right side portion thereof and a connector is inserted in the connector insertion portion 37A through the insertion hole 51D. As illustrated in FIG. 10, the first casing 51 includes a second communication hole 51E in a rear surface thereof. The second communication hole 51E is communicated with a space in which the reset button 46 is disposed. An operation pin is inserted in the second communication hole 51E from an outside to press the reset button 46 and accordingly, the control section 44 executes a reset process to return the device 40 to an initial state.

As illustrated in FIGS. 7, 8, 11, and 12, the second casing 52 includes a cover 52A, the two fitting portions 52B, two first projecting portions 52C, and a second projecting portion 52D. The cover 52A extends over the exposed contacts 41B. The cover 52A includes a ceiling wall 52A1 that covers the contacts 41B from above and a right wall 52A2 (another example of the positioning portion) that covers the contacts 41B from a right side. The cover 52A does not cover the contacts 41B from a front side and a left side and has an opening 52A3 that has a substantially L-shape and opens frontward and leftward. With the cover 52A including the right wall 52A2 and the opening 52A3, the packaging material 20 can be inserted in the opening 52A3 and positioned with respect to the device with being contacted with the right wall 52A2 when mounting the packaging material 20 in the device 40. Since the cover 52A is open leftward through the opening 52A3, the packaging materials 20 having various sizes (different lengths in the short-side direction) can be fitted in the opening 52A3.

As illustrated in FIGS. 7 and 8, the fitting portion 52B extends downward from the cover 52A and fitted to the bottom portion 51A of the first casing 51. The fitting portion 52B includes the protrusion 52B1 at the extending end thereof and the protrusion 52B1 is fitted to the protrusion 51A1 of the bottom portion 51A. The protrusion 52B1 of the fitting portion 52B is disposed below the protrusion 51A1 (FIG. 7, referred to as a first position) when the second casing 52 moves downward. In such a state, the packaging material 20 is held between the first casing 51 and the second casing 52 and the fitting portion 52B is in a holding state. The protrusion 52B1 of the fitting portion 52B is disposed on the protrusion 51A1 (FIG. 8, referred to as a second position) when the second casing 52 moves upward. In such a state, the packaging material 20 is not held between the first casing 51 and the second casing 52 and the fitting portion 52B is in a non-holding state. The fitting portion 52B is fitted in and connected to the bottom portion 51A of the first casing 51 in the first position and the second position.

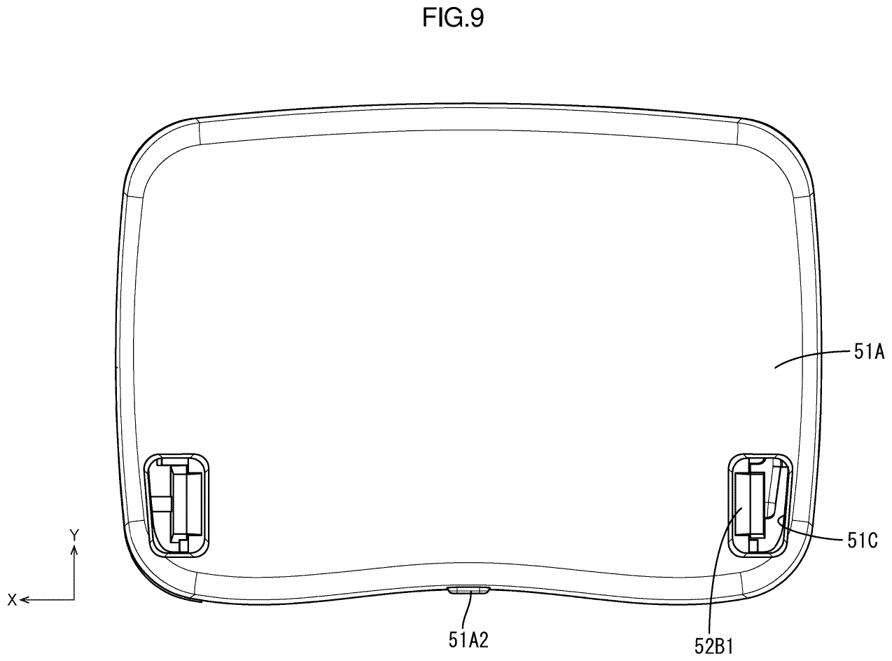

With the cover 52A being pushed downward from above, the protrusion 52B1 moves downward from above to below the protrusion 51A1 and the fitting portion 52B moves from the second position to the first position. With an operation pin being inserted through the first communication hole 51C from the outside and the protrusion 52B1 being operated with the operation pin, the fitting portion 52B moves from the first position to the second position. More in detail, as illustrated in FIG. 9, the opening detection device 40 is disposed such that the bottom portion 51A faces upward. In this state, the operation pin is inserted in the first communication hole 51C from the outside and the protrusion 52B1 is pushed downward (toward the upper portion 51B) with being moved in the right-left direction (for example, in a direction illustrated with a blank arrow in FIG. 7) with the operation pin. Then, the protrusion 52B1 slides along a sloped surface 51A1S of the protrusion 51A1. Thus, the fitting portion 52B easily moves from the first position to the second position. Accordingly, to change the state of the packaging material 20 from the holding state (the first position) to the non-holding state (the second position), the protrusion 52B1 is necessarily moved with using the operation pin and cannot be operated directly with a hand. Therefore, an erroneous operation is less likely to occur. In the holding state, the packaging material 20 can be held more stably. In the non-holding state (the second position), the sloped surface 51A1S of the protrusion 51A1 is covered by the protrusion 52B1. Therefore, it is difficult to move the protrusion 52B1 with using the operation pin from the outside and completely detach the second casing 52 from the first casing 51. With such a configuration, the second casing 52 is connected to the first casing 51 so as to be movable in the upper-bottom direction (the Z-axis direction) but not to be completely detached from the first casing 51. The phrase that the first communication hole 51C is communicated with the fitting portion 52B means that the first communication hole 51C is communicated with a space where the fitting portion 52B is disposed in the holding state (the first position).

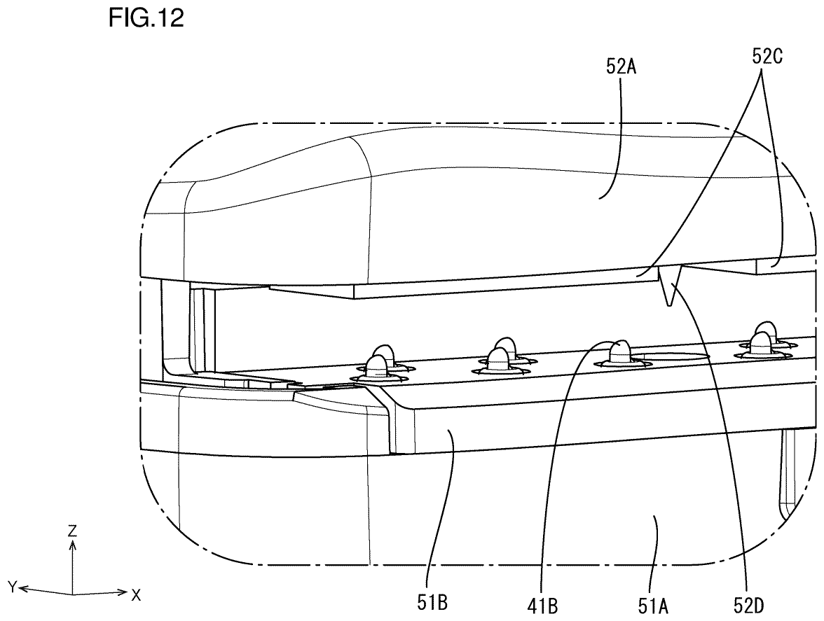

The first projecting portions 52C are made of a soft material having flexibility. As illustrated in FIGS. 11 and 12, the first projecting portions 52C project downward from the cover 52A. The first projecting portion 52C has a laterally elongated cubic shape and two first projecting portions 52C are arranged at an interval with respect to the right-left direction (the X-axis direction). The second projecting portion 52D is made of a hard material and has a tapered bar shape. The second projecting portion 52D is between the two first projecting portions 52C. With the packaging material 20 being mounted in the opening detection device 40 (FIG. 2), projecting surfaces (a lower surface) of the first projecting portions 52C and the distal end of the second projecting portion 52D are press-contacted with the surface of a body member 21 of the packaging material 20.

With the first projecting portions 52C and the second projecting portion 52D being formed, the holding force for holding the packaging material 20 can be increased. The first projecting portions 52C and the second projecting portion 52D are not contacted with the circuit pattern 32 of the packaging material 20 and are not electrically conductive. Therefore, the holding force can be increased without adversely affecting the opening detection. The materials and the shapes of the first projecting portions 52C and the second projecting portion 52D may be altered as appropriate as long as the holding force can be increased by the anti-slip effect. For example, the first projecting portions 52C and the second projecting portion 52D may be made of elastic material, adhesive material, and high friction material. Both of the first projecting portion 52C and the second projecting portion 52D may not be necessarily provided but one of the first projecting portion 52C and the second projecting portion 52D may be provided.

With the casing 50 having the above configuration, the packaging material 20 can be mounted in the opening detection device 40 easily and firmly without using extra fixing member. The mounting and detaching of the packaging material 20 can be performed easily by the movement of the second casing 52.

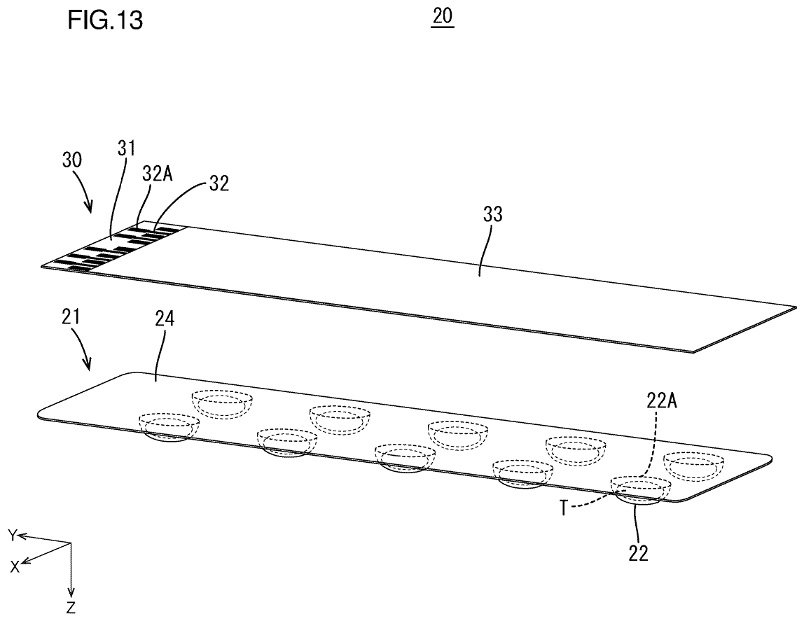

As illustrated in FIG. 13, the packaging material 20 includes the body member 21 for storing medicine T such as medical tablets and an opening detection sheet 30 that is attached to the body member 21. In this embodiment, the body member 21 is a known press through package (PTP) but may be other type of member besides PTP as long as it can store the objects. The body member 21 has a parallelopiped (rectangular) plan view shape as a whole and includes a sheet-shaped container and a cover 24. The sheet-shaped container includes storing portions 22 in each of which the medicine T is stored. The cover 24 is attached to the sheet-shaped container and covers openings 22A of the storing portions 22 and the storing portions 22 are sealed.

As illustrated in FIG. 13, the opening detection sheet 30 has a plan view shape and a plan view size according to the body member 21 and is attached to the cover 24 of the body member 21. The opening detection sheet 30 is a composite member of a sheet form and includes a base 31, the circuit pattern 32, and a protection layer 33 that are disposed on top of each other from the body member 21 side. The circuit pattern 32 includes traces that are formed by printing on the base 31 with electrically conductive ink. The circuit pattern 32 is formed to include the same number of current flow paths as that of the storing portions 22. A portion of each current flow path overlaps the opening 22A of the storing portion 22 in a plan view. The protection layer 33 is made of resin material and covers the circuit pattern 32. The protection layer 33 is formed on a surface except for the ends 32A of the traces of the circuit pattern 32. In other words, the protection layer 33 does not cover the ends 32A of the circuit pattern 32 and the ends 32A are exposed to the outside.

The packaging material 20 is attached to the opening detection device 40 (FIG. 2) such that the surface (a first surface) of the opening detection sheet 30 is opposite the first casing 51 and the surface (a second surface) of the body member 21 is opposite the second casing 52. With the packaging material 20 being attached to the device, the ends 32A of the circuit pattern 32 and the contacts 41B of the contact portions 41 are contacted with each other and electrically conductive.

According to the packaging material 20 having the above configuration, with the medicine T being pushed out from the storing portion 22, the opening detection sheet 30 is broken together with the cover 24. Accordingly, the circuit pattern 32 that overlaps the storing portion 22 in a plan view is broken. As a result, a voltage value generated in the circuit pattern 32 changes before and after the breakage (before and after the medicine T is pushed out). Thus, as previously described, it can be determined by the control section 44 of the opening detection device 40 whether the packaging material 20 is in an opened state or unopened state.

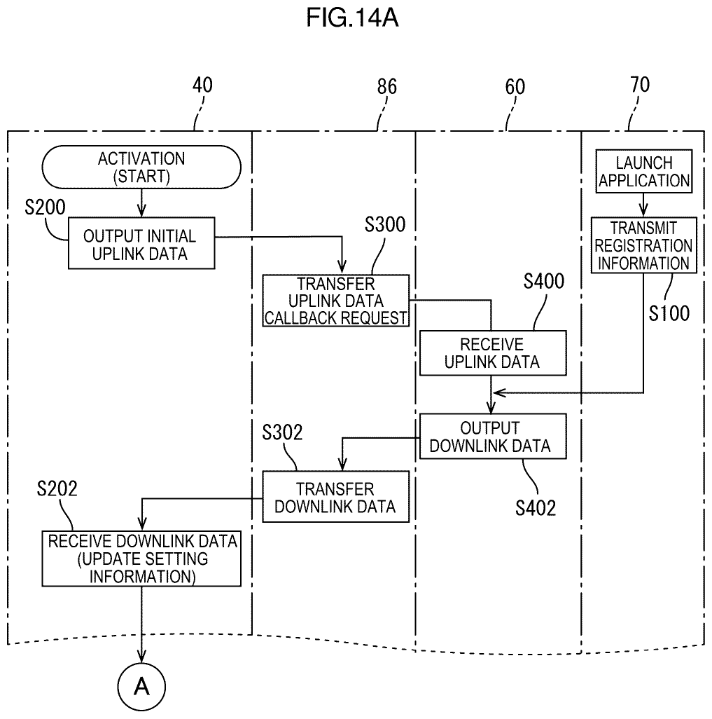

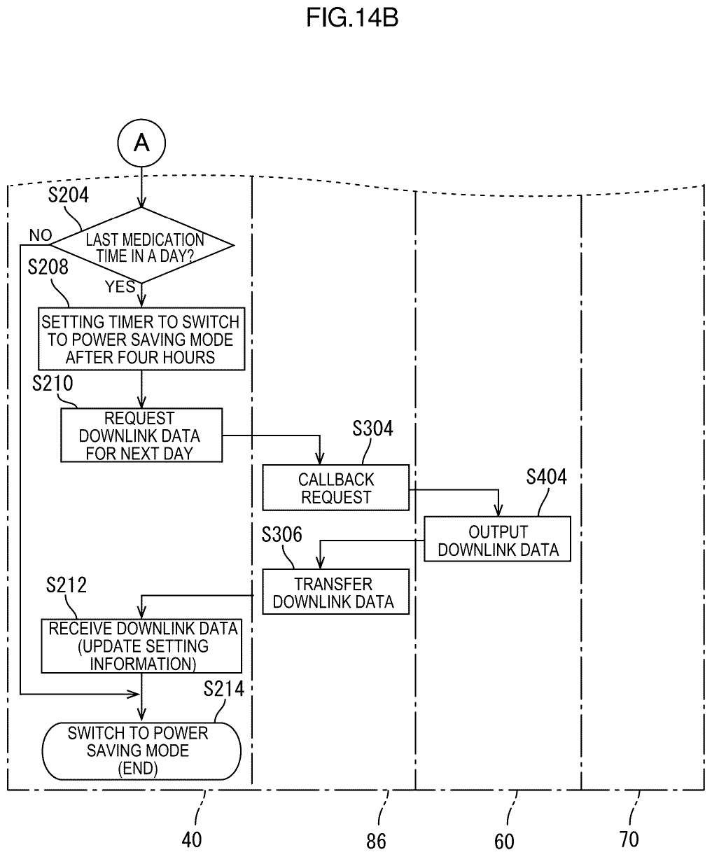

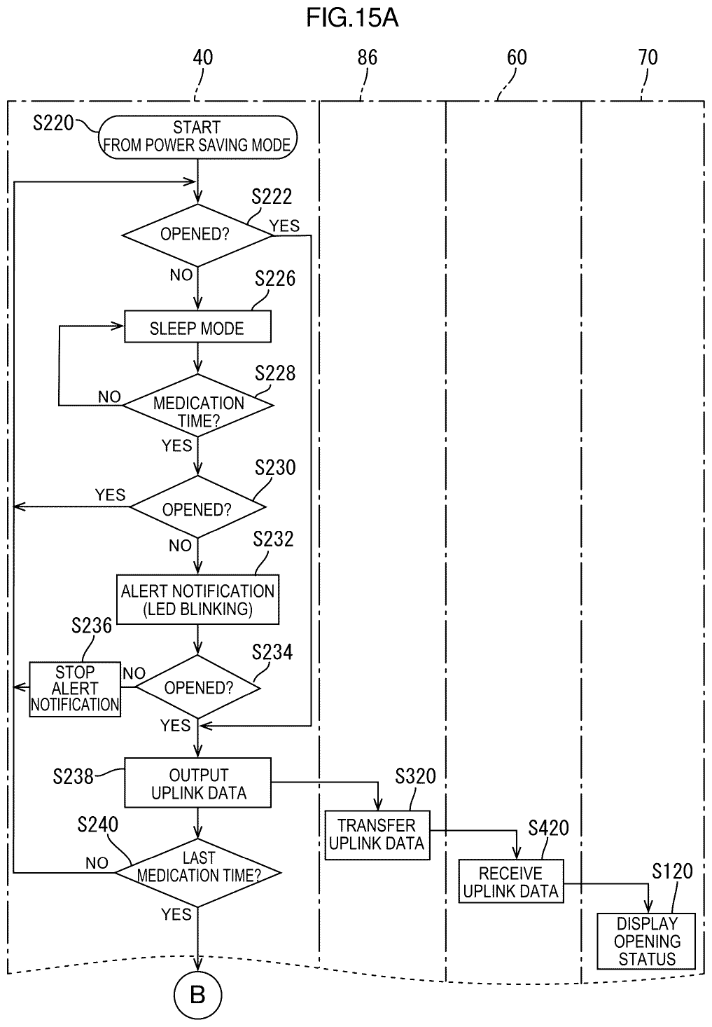

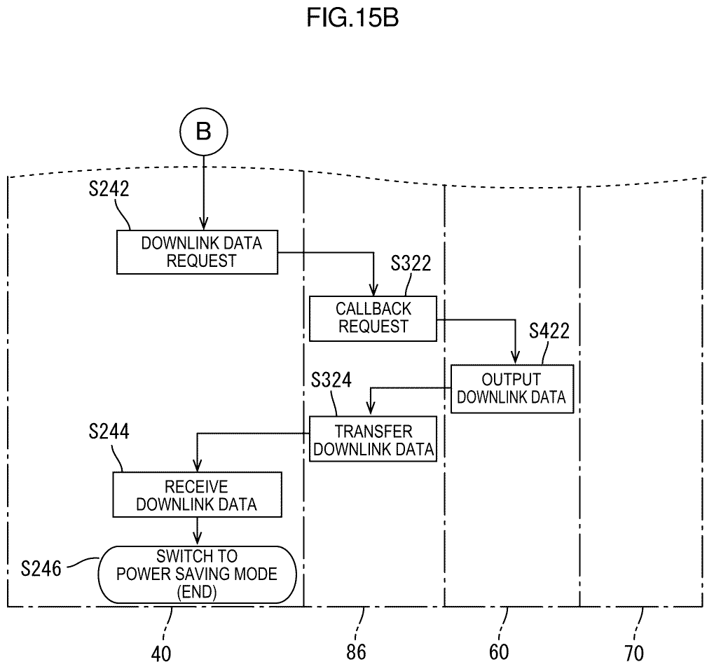

Next, a specific processing of the opening management system 10 having the above-described configuration will be described with reference to FIGS. 14A, 14B, 15A, 15B. In the following, a transmission flow of information sequentially from the information processing terminal 70 to the opening detection device 40 via the management server 60 and the communication server 86 is defined as downlink communication. A transmission flow of information sequentially from the opening detection device 40 to the information processing terminal 70 via the communication server 86 and the management server 60 is defined as uplink communication.

A user launches an application stored in the information processing terminal 70 and inputs registration information of the opening detection device 40. When inputting the registration information, a user inputs the ID information on the casing 50 of the opening detection device 40 to the information processing terminal 70 or the information processing terminal 70 directly reads the ID information. Accordingly, the management server 60 records the corresponding relation of the ID information of the opening detection device 40 and the identification information of the information processing terminal 70. The ID information may be on the casing 50 of the opening detection device 40 in plaintext format or a ciphered code representing the ID information may be on the casing 50. The ciphered code may be decoded by the management server 60 to obtain the ID information. The ID information of the opening detection device 40 may be unique information of each opening detection device 40 or each information processing terminal 70. The identification information of the information processing terminal 70 may be unique information of the information processing terminal 70 or unique information of a user who uses the information processing terminal 70.

As illustrated in FIG. 14A, the input registration information is transmitted to the management server 60 from the information processing terminal 70 via a downlink (S100). Specifically, the registration information includes information representing medication time to take medicine T (one example of detection time), information representing opening detection frequency of detection with the opening detection device 40, information representing setting of a notification type with the opening detection device 40, information representing transmission frequency of transmitting the opening detection result from the opening detection device 40, and information representing request frequency of transmission of setting information from the management server 60. The registration information transmitted via a downlink to the management server 60 is linked to the ID information of the opening detection device 40 and the identification information of the information processing terminal 70.

The registration information relating the medication time includes each medication time if the medicine is to be taken several times a day. For example, if the medicine is to be taken three times a day after meal, the medication times include the time after breakfast, the time after lunch, and the time after supper. The opening detection frequency is for setting one of regular detection of regularly detecting at every predetermined timing (referred to as regular detection, hereinafter) or continuous detection (referred to as real time detection, hereinafter), and further for setting a time interval (for example, five minutes, ten minutes, fifteen minutes, twenty minutes, thirty minutes, sixty minutes, one hundred and twenty minutes) with the regular detection being performed. With performing the regular detection, power consumption can be reduced compared to performing the real time detection and as the time interval of the regular detection is set to be longer, the power consumption can be further reduced. In this embodiment, the operations of the regular detection will be described specifically.

The setting of a notification type represents setting of a lighting pattern of the LED 45 when the medicine is not taken (not opened) and can be selected from various types of patterns having different turned-on periods with steady light (blinking light). The transmission frequency of transmitting the opening detection result from the opening detection device 40 represents setting of whether or not to perform information transmission (uplink) from the opening detection device 40 only when the opening detection result changes. If the transmission is performed only when the opening detection result changes, the data transmission frequency with the opening detection device 40 can be reduced and the power consumption is also reduced. In this embodiment, the operations with performing data transmission only when the opening detection result changes will be specifically described.

The request frequency of transmission of setting information from the management server 60 (referred to as regular downlink frequency, hereinafter) is for setting the number of transmission to be once a day (for example, after the medication time after supper) or several times a day (for example, after each medication times after breakfast, after lunch, after supper). By adjusting the regular downlink frequency, the setting information of the opening detection device 40 can be updated and the power consumption necessary for the updating can be adjusted to be as small as possible. In this embodiment, the operations when the regular downlink frequency is set to be after each medication time will be specifically described.

As illustrated in FIG. 14A, with the opening detection device 40 being activated, initial data is transmitted from the opening detection device 40 via an uplink (S200). Data transmitted from the opening detection device 40 via an uplink (uplink data) includes ID information, opening time, opening detection result (a determination result regarding the opening status of each storing portion 22 made by the control section 44), residual energy of the rechargeable battery 47, connection or disconnection of the circuit pattern 32 of the packaging material 20 and the connection portion 41, and an operation state of an independent mode. The uplink data that is set when the opening detection device 40 is activated or the reset button 46 is turned on is defined as the initial data. The uplink data includes the ID information of the opening detection device 40. Therefore, even if there are several opening detection devices 40 (there are several packaging materials 20 (several kinds of medicines T)), the opening detection is performed for each opening detection device 40 and each opening detection device 40 can inform the opening status.

The independent mode is operated when downlink data, which will be described later, is not transmitted to the opening detection device 40 due to the poor connection. In the independent mode, the uplink data is not transmitted at the data transmission frequency (transmission frequency of transmitting the opening detection result) that is set based on the registration information input through an application software but transmitted at the regular interval determined by a timer included in the control section 44 of the opening detection device 40. In the independent mode, even if the downlink data is not normally transmitted to the opening detection device 40 due to the poor connection, the uplink data can be transmitted from the opening detection device 40. However, since the present time included in the downlink data is not transmitted to the opening detection device 40, the LED 45 is not turned on with blinking light to make alert notification and inform that the medicine is not taken when the independent mode is operated.

As illustrated in FIG. 14A, if the communication server 86 receives the uplink data from the opening detection device 40, the communication server 86 transfers the uplink data to the management server 60 and requests the management server 60 callback of (to transmit) the setting information (S300). The management server 60 receives the uplink data that is transferred from the communication server 86 (S400). The information generation section 62 of the management server 60 generates the setting information based on the registration information that is transmitted from the information processing terminal 70 at step S100. The setting information is used when the opening detection device 40 performs the predefined process regarding the opening detection. The setting information (referred to as the downlink data, hereinafter) is output (transmitted) in response to the callback request of the communication server 86 (S402). In this embodiment, the downlink data is the setting information that is generated as 64-bit data and includes the registration information previously described and the present time.

As illustrated in FIG. 14A, the communication server 86 receives the downlink data from the management server 60 and transfers the downlink data to the opening detection device 40 (S302). The downlink data is rewritten in the storing section 48 of the opening detection device 40 and the setting information of the opening detection device 40 is updated (S202). Therefore, after the updating, the control section 44 of the opening detection device 40 performs the predefined process regarding the opening detection based on the updated setting information.

Next, as illustrated in FIG. 14B, the control section 44 of the opening detection device 40 determines whether it is the last medication time in a day (for example, it is the third time if the frequency is three times a day) (S204). If it is determined to be the last medication time (YES at S204), the control section 44 sets a timer to switch the operation mode to a power saving mode (night mode) after predetermined period has passed (for example, after four hours) (S208). The night mode is one of the power saving modes and in the night mode, the operation of a stand-by mode, which will be described later, is performed and the regular detection is not performed. In the power saving mode, the opening detection device 40 is set in a stand-by state while no medication is necessary and the opening detection is not necessary such as during night time, and the power consumption can be reduced by not performing the opening detection.

After setting the timer, if the downlink data for the next day is requested by the opening detection device 40 (S210), the callback request is output from the communication server 86 (S304) and the downlink data is output (transmitted) from the management server 60 (S404). The downlink data is transmitted to the opening detection device 40 via the communication server 86 (S306). The downlink data is rewritten in the storing section 48 of the opening detection device 40 and the setting information of the opening detection device 40 is updated (S212). After the time set by the timer at step S206 passes, the operation mode of the opening detection device 40 is switched to the power saving mode and becomes in the stand-by state (S214). On the other hand, it is not determined to be the last medication time at step S204 (NO at S204), the control section 44 switches the operation mode of the opening detection device 40 to the power saving mode (the stand-by mode) (S214). The stand-by mode is one of the power saving modes. In the stand-by mode, the opening detection (supplying a current to the connection portion 41 of the opening detection device 40, detecting a voltage generated in the circuit pattern 32 of the packaging material 20, and determining the opening status) is not performed.

After the operation mode is switched to the power saving mode (S214 in FIG. 14B), as illustrated in FIG. 15A, the opening detection device 40 is started in the power saving mode at a predetermined timing (for example, in performing the regular detection, at an activation timing in response to the timer of the control section 44 based on the detection interval included in the downlink data) (S220). The control section 44 determines whether each storing portion 22 is opened (S222) and if determining that the storing portion 22 is opened, the control section 44 outputs (transmits) the uplink data (S238). The uplink data is transmitted to the management server 60 (S420) via the communication server 86 (S320) and the opening status is displayed on the information processing terminal 70 (S120). On the other hand, if it is determined that the storing portion 22 is not opened at step S222, the control section 44 switches the operation mode of the opening detection device 40 to the power saving mode (the stand-by mode) (S226).

Next, if determining that the present time is on or after the medication time (YES at S228), the control section 44 releases the power saving mode (the stand-by mode) (S229) and performs the opening detection process again (S230). If it is determined that the opening status is an unopened state (NO at S230), the packaging material 20 is not opened (the medicine T is not taken out) although it is past the medication time. The LED 45 is turned on with blinking light to make alert notification (S232). On the other hand, if it is determined that the opening status is an opened state (YES at S230), the process returns to step S222.

Next, the control section 44 performs the opening detection process again after the predefined time interval of the regular detection (S234). If it is determined that the opening status is an unopened state (NO at S234), the opening status does not change from the unopened state, which is the previous opening detection result (NO at S230), the uplink data is not output (transmitted) and the alert notification is stopped after a predefined time passes (S236) and the process returns to step S222. On the other hand, if it is determined that the opening status is the opened state (YES at S234), the opening status changes from the unopened state, which is the previous opening detection result (NO at S230), the uplink data is output (transmitted) to the communication server 86 (S238). The uplink data includes data representing the opened time that corresponds to the present time when the control section 44 of the opening detection device 40 detects the opened state. The uplink data is transmitted to the management server 60 (S420) via the communication server 86 (S320) as previously described and the opening status is displayed on the information processing terminal 70 (S120). The management server 60 transmits the uplink data to the information processing terminal 70 and the information processing terminal 70 displays the opening status, which at least includes the opened time, based on the uplink data. The management server 60 obtains the ID information of the opening detection device 40, which is included in the uplink data, and transfers the uplink data to the information processing terminal 70 from which the registration information corresponding to the obtained ID information is inputted.

After outputting the uplink data (S238), the control section 44 of the opening detection device 40 determines whether the present time is on or after the last medication time (S240). The last medication time is the latest time among the multiple medication times included in the downlink data. The last medication time is the last time that the medicine T must be taken and is the time after the predetermined time past the medication time. The medicine T is required to be taken on or after the medication time and at least before the last medication time. Therefore, the packaging material 20 is required to be opened on or after the medication time and before the last medication time. If the present time is not on or after the last medication time (NO at S240), the process returns to step S222 and the process regarding the opening detection that is previously described will be repeatedly performed.

If the present time is on or after the last medication time (YES at S240), as illustrated in FIG. 15B, the opening detection device 40 requests the downlink data (S242) and the callback request is output from the communication server 86 (S322) and the downlink data is output (transmitted) from the management server 60 (S422). The downlink data is transmitted to the opening detection device 40 via the communication server 86 (S324). The downlink data is rewritten in the storing section 48 of the opening detection device 40 and the setting information of the opening detection device 40 is updated (S244). After the updating, the operation mode of the opening detection device 40 is switched to the power saving mode (S246).

### Second Embodiment

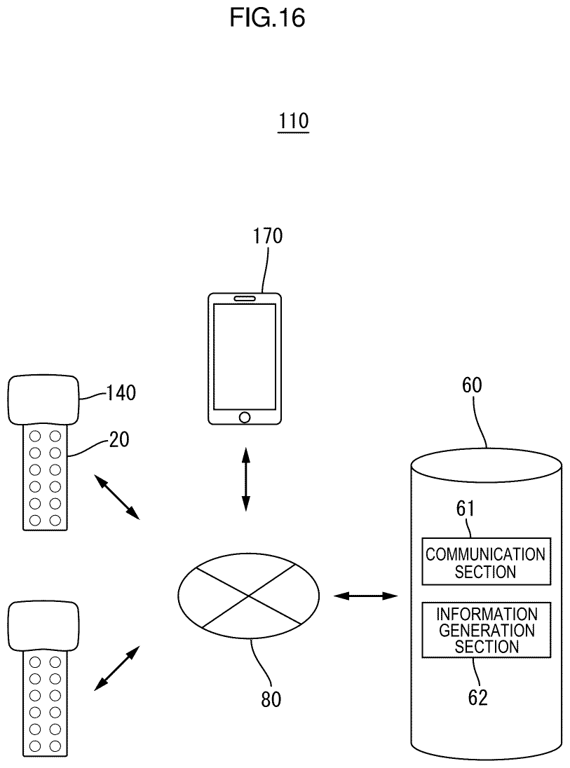

An opening management system 110 according to a second embodiment will be described with reference to FIG. 16. The configuration, operations, and effects of the second embodiment that are similar to those of the first embodiment will not be described.

In the opening management system 110, opening detection devices 140 are connected to an information processing terminal 170 with Bluetooth low energy (BLE, registered trademark) so as to communicate each other. The opening detection device 140 includes a BLE communication module and the information processing terminal 170 has a BLE function. With such a configuration, the opening detection device 140 is paired to the information processing terminal 170 and the opening detection device 140 can communicate with the management server 60 that is connected to the communication network 80 via the information processing terminal 170. Accordingly, even with the LPWA communication being not available (for example, in the area without having the base station 85), the opening detection and the notification based on the opening status can be performed.

### Other Embodiments

The present invention is not limited to the embodiments described above with reference to the drawings. For example, the following embodiments may be included in the technical scope of the present invention.
(1) The shape of the casing 50 illustrated in the drawings is one example and may be altered as necessary.
(2) The shape of the packaging material 20 and the number of the storing portions 22 and the interval that are illustrated in the drawings are one examples and may be altered as necessary.

### EXAMPLES OF USING UPLINK DATA

As previously described, the management server 60 transfers the uplink data to the information processing terminal 70; however, the management server 60 may transmit various kinds of processed data that is generated with performing information processing based on the uplink data to the information processing terminal 70. Furthermore, the management server 60 may transmit the uplink data or the processed data to a terminal other than the information processing terminal 70 to which the registration information is input.

The uplink data and the processed data may be transmitted to a terminal that is used by a person who takes medicine T packed in the packaging material 20, a terminal in the hospital that provides the medicine T, a terminal used by a pharmaceutical manufacturer that produces the medicine T, or a terminal that is used by a distributer of the medicine T. The management server 60 is preferably configured such that the uplink data and the processed data cannot be accessed by external devices and the uplink data and the processed data are transmitted only to reliable receivers. The management server 60 may cipher the uplink data and the processed data and store them such that only the reliable receivers can decode.

As previously described, the management server 60 may perform the following information processing based on the uplink data. The management server 60 may create an opening time data base that includes a plurality of registration information input via the information processing terminal 70 and a plurality of opening times that are represented by the uplink data. Furthermore, the management server 60 may compare the medication time represented by the registration information and the opening time and determine whether the medicine is appropriately taken and transmit the processed data representing the determination result to the terminal. Such processed data is helpful to a person who prescribes the medicine T such as a doctor and a pharmacist and also helpful to a person who takes the medicine T.

The management server 60 may generate the processed data by statically processing the opening times stored in the opening time data base. Specifically, the management server 60 may statistically process the opening times for every attribute (for example, age and sex) of the persons who take medicine T or may statistically process the opening times for every kind of the medicines T. Furthermore, the management server 60 may create a prediction model for predicting the medication status of the medicine T through the machine learning with using the opening times stored in the opening time data base as training data. The management server 60 may transmit the data representing the medication status that is predicted according to the prediction model to each terminal as the processed data.

The packaging material 20 is not necessarily for packaging the medicine T but may be for packaging any objects as long as the packaging material is to be opened and the objects are used or consumed. Furthermore, the packaging material may be any packaging material as long as the opening detection device 40 can be attached to and the objects are not necessarily sealed in the packaging material. The packaging material may be a sealing material that is to be attached to a container storing an object and the stored object. Such packaging material may be broken by taking the stored object out of the container. For example, the packaging material may be a sealing material attached to a file folder as a container and a book or a booklet as a stored object. For example, with the present invention being applied to an object having a limited expiration date such as a food, it can be confirmed whether the object is consumed at a proper timing based on the opening time represented by the uplink data. With the present invention being applied to an object having an unlimited expiration date, the consumption condition of the object can be obtained based on the opening time represented by the uplink data. Therefore, helpful information can be provided to the suppliers or the producers of the objects.

### EXPLANATION OF SYMBOLS

10: opening management system, 20: packaging material, 40, 140: opening detection device, 43: communication section, 44: control section (detection section), 45: LED (informing section), 61: communication section, 62: information generation section, T: medicine

1. An opening management system comprising:
a communication section configured to communicate with an opening detection device that is attached to a packaging material and detects an opening status of the packaging material; and
an information generation section configured to generate setting information with which a predefined process regarding opening detection is performed by the opening detection device.

2. The opening management system according to claim 1, wherein
the setting information includes detection time at which the opening detection device performs the opening detection, and
the predetermined process includes detecting the opening status of the packaging material on or after the detection time and informing if the opening status is an unopened state.

3. The opening management system according to claim 1 or claim 2, wherein
the setting information includes detection frequency at which the opening detection device performs the opening detection, and
the predetermined process includes detecting the opening status of the packaging material at the detection frequency and informing if the opening status is an unopened state.

4. The opening management system according to claim 3, wherein regular detection in which detection is performed every predetermined time period or continuous detection is set as the detection frequency.

5. The opening management system according to any one of claims 1 to 4, wherein the setting information includes request frequency at which the opening detection device requests transmission of the setting information.

6. The opening management system according to claim 5, wherein when the communication section receives a request of the transmission of the setting information from the opening detection device, the communication section transmits the setting information to the opening detection device.

7. The opening management system according to any one of claims 1 to 6, wherein the setting information includes transmission frequency at which the opening detection device transmits the opening status to the communication section.

8. The opening management system according to any one of claims 1 to 7, wherein
the opening detection device includes a plurality of opening detection devices each of which has ID information,
the setting information includes the ID information, and
the communication section is configured to communicate with each of the plurality of opening detection devices.

9. The opening management system according to any one of claims 1 to 8, wherein medicine is stored in the packaging material.

10. An opening detection device comprising:
a communication section configured to communicate with the opening management system according to any one of claims 1 to 9; and
a detection section configured to detect the opening status of the packaging material.

11. The opening detection device according to claim 10, further comprising an informing section configured to inform when a detection result of the detection section is an unopened state.

12. The opening detection device according to claim 10 or claim 11, wherein the detection section is configured to detect the opening status of the packaging material on or after detection time included in the setting information.
